(19) United States
(12) Patent Application Publication
(10) Pub. No.: US 2023/0148846 A1
(43) Pub. Date: May 18, 2023

(54) Single Piece Stamped Flexure

(71) Applicant: GREENE GROUP INDUSTRIES, LLC, Oceanside, CA (US)

(72) Inventors: Frank Staender, Encinitas, CA (US); Steven S. Jacobs, Vista, CA (US); Stephen J. Waszak, Escondido, CA (US)

(73) Assignee: GREENE GROUP INDUSTRIES, LLC, Oceanside, CA (US)

(21) Appl. No.: 18/056,532

(22) Filed: Nov. 17, 2022

Related U.S. Application Data
(60) Provisional application No. 63/280,203, filed on Nov. 17, 2021.

(51) Int. Cl. A61B 1/008; A61B 34/30; A61B 1/01

(52) U.S. Cl. CPC A61B 1/008; A61B 34/30; A61B 1/01; A61B 2034/306

(57) ABSTRACT

An instrument flexible guide is made of an elongated single piece of a material or alloy by stamping cutouts interposed by single narrow remaining hinge portions. The cutouts and narrow hinge portions are in different positions. The single piece is rolled into a tubular shape, with curved rings joined by the hinges. Spaced rings are bent inward as guides for flexible instruments. Seams are welded or left unwelded.

This application claims priority under 35 U.S.C. § 119(e) to U.S. Provisional Patent Application No. 63/280,203, filed Nov. 17, 2021, the entirety of which is incorporated by reference herein.

### SUMMARY OF THE INVENTION

The invention provides a flexible stamped flexure structure. The new invention has many advantages such as reduced cost, lending itself to single use applications, more easily scalable for higher volumes, and is one piece, less complex, more reliable, less manufacturing steps, less reliance on skilled labor. Moreover, the previous devices have multiple pieces, requiring assembly and thus introducing error potential and risk at each assembly.

The new flexible stamped flexure is useful in medical devices. Previously known hinge assemblies, link assemblies and snake wrists have high costs and are not attractive for single use applications.

The new single piece stamped flexures are such as those used in medical devices that are typically called hinge assemblies, link assemblies or snake wrists, the new structure offers a low-cost alternative to the multipiece assemblies currently in the field. The invention, in addition to the significant manufacturing cost reduction, utilizes an easily scalable manufacturing process. The stamped flexure performance is adjustable via application of various geometries and materials. The function and performance of the new flexure can be altered in order to make it more or less spring-like, increase or decrease the bend radius, and increase or reduce the number of repeating segments. These alterations are done by changing one or more of the properties of the type of material/alloy used, the thickness of the material, the spacing of the cutouts, the shape of the cutouts, the clocking of the cutouts relative to one another, the use of various combinations of cutouts having different positions in the sequence of the repeating segments, and the diameters of the flexures. The type of heat treatment or similar alloy alterations can also be altered to adjust the performance of the device.

This invention can be used with and in various manual and robotic surgical and endoscopic instruments that employ what can be known as:
Snake wrist
Snake
Hinge assembly
Link assembly
Flexure

The flexible result and direction can be controlled by cables that are manipulated manually (i.e. handheld units) or remotely/motorized, for example in robotic assisted surgery and endoscopy.

These and further and other objects and features of the invention are apparent in the disclosure, which includes the above and ongoing written specification, with the drawings.

### BRIEF DESCRIPTION OF THE DRAWINGS

FIGS. 1-3 show the single piece stamped hinge assembly invention.

FIG. 4 is an end view of the stamped flexure shown in FIGS. 1-3.

FIG. 5 is a perspective side and end view of the new stamped flexure shown in FIGS. 1-4.

FIG. 6 is a detail of interposed cable guidance sections of the new stamped flexure.

FIGS. 7-9 show currently used medical devices having multi-piece hinge assembly.

### DETAILED DESCRIPTION

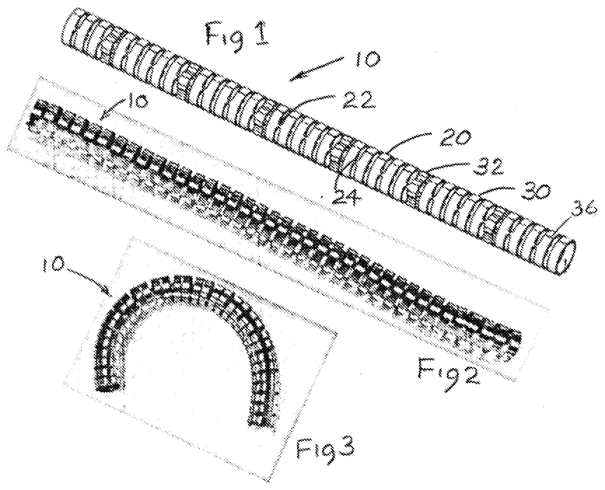

FIG. 1 shows a new single piece stamped flexure, to replace those used in medical devices that are typically called hinge assemblies, link assemblies or snake wrists. The new single piece stamped flexure 10 offers a low-cost alternative to the multipiece assemblies currently in the field. The invention, in addition to the significant manufacturing cost reduction, utilizes an easily scalable manufacturing process.

FIG. 2 shows a new stamped flexure 10 that has been slightly bent 12. FIG. 3 shows a new stamped flexure 10 that has been curved to a new shape 14.

The stamped flexure performance is adjustable via application of various geometries and materials. The function/performance of the flexure can be altered in order to make it more or less spring-like, increase or decrease the bend radius, and increase or reduce the number of repeating segments. These alterations are done by changing one or more of the properties of the type of material/alloy used, the thickness of the material, the spacing of the cutouts 20 and the remainder hinges 22 at the ends 24 of the cutouts that form connected segments 30. The shape of the cutouts, the circular position clocking of the cutouts relative to one another, the use of various combinations of cutouts having different positions in the sequence of the repeating segments, and the diameter of the flexure are controlled in the manufacture. The type of heat treatment or similar alloy alterations can also be altered to adjust the performance of the device. Integrally connected segments 32 have inward extensions 34 that guide and support instruments inside the stamped flexure 10.

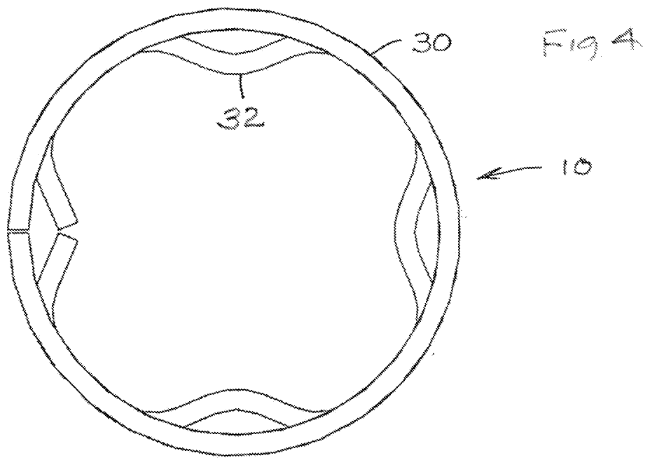

In FIG. 4 the inward extensions 34 for guiding and centering inserted instruments are shown.

The special guiding ring sections 32 and inward extensions 34 are interposed between the regular spaced and connected ring sections 30 as shown in FIGS. 1, 5 and 6.

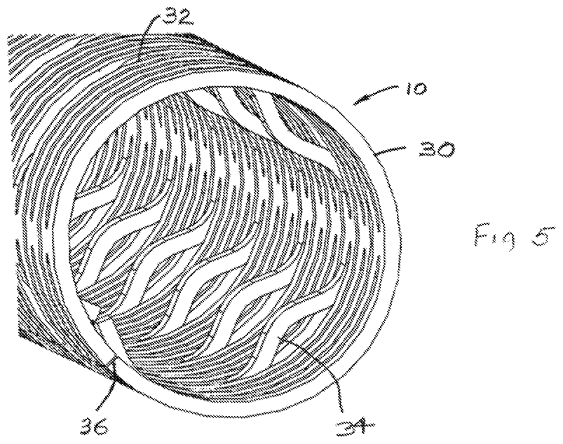

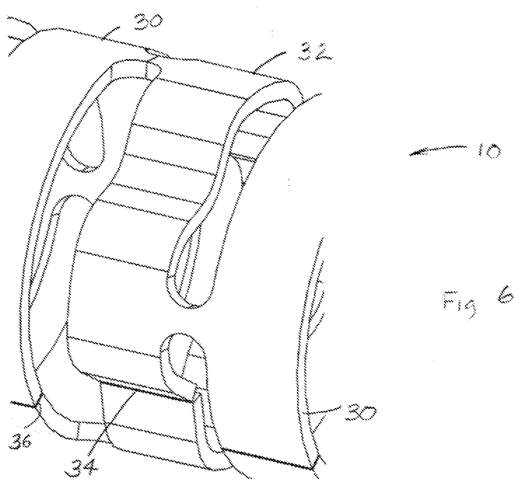

The end view of FIG. 4 along with the details of FIGS. 5 and 6 accentuate the formed cable vias. As with all features, these can be adjusted in size and location throughout the stamped flexure to meet functional needs.

The stamped medical hinged devices are stamped and rotated into the tubular shape of flexure 10 with the dies, completing the structures without steps that use time and are expensive and present risks.

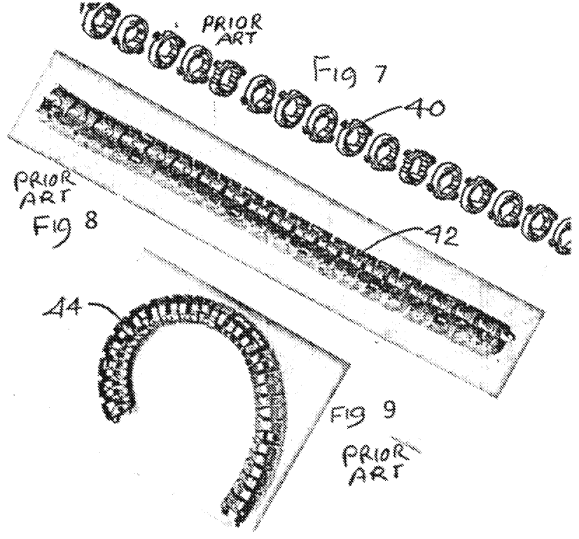

FIGS. 7-9 show prior art assembled multi-pieces forming hinge assembly medical devices. Individual elements 40 are shown in FIG. 7. Individual elements 40 for guiding are mechanically joined together as shown in FIG. 8, so that the mechanically joined 42 individual elements 40 may be moved into curves 44, as shown in FIG. 9.

The invention is new and unobvious from prior art devices.

While the invention has been described with reference to specific embodiments, modifications and variations of the invention may be constructed without departing from the scope of the invention.

We claim:

1. Apparatus comprising:
a single piece stamped flexure having spaced cutouts forming repeating segments that are rolled into a tubular shape having a seam.

2. The apparatus of claim 1 wherein the repeating segments form rings and hinges between the spaced cutouts.

3. The apparatus of claim 1 wherein the spaced cutouts are clocked at different positions in the single piece stamped flexure.

4. The apparatus of claim 1 wherein the flexure is altered by changing bend radius by changing spaced cutouts.

5. The apparatus of claim 1 wherein the flexure is altered by changing clocking of the spaced cutouts relative to one another.

6. The apparatus of claim 1 wherein the flexure is altered by changing the shape of the spaced cutouts.

7. The apparatus of claim 1 wherein the flexure is altered by combinations of different cutouts having different positions in a sequence of repeating segments.

8. The apparatus of claim 1 wherein the flexure has flexibly controlled by a type of material or alloy used, thickness of the material or alloy used.

9. The apparatus of claim 1 wherein the flexure is controlled by diameters of the flexure.

10. The apparatus of claim 1 further comprising inward extensions adapted for guiding and centering inserted instruments.

11. The apparatus of claim 10 wherein the inward extensions are interposed between the rings formed by the repeating segments.

12. Apparatus comprising a single piece stamped flexures formed from a material or alloy and having spaced cutouts and repeating segments connected with narrow hinge portions, wherein the repeating segments are formed into rings connected by the narrow hinge portions by rolling the single piece into a tubular shape having the seam between abutting portions of the repeating segments.

13. The apparatus of claim 12 wherein the cutouts are clocked in different positions and the hinge portions are in different positions around the tubular shape for providing flexibility to the flexure.

14. The apparatus of claim 13 wherein some spaced rings have inward extensions interposed between other spaced and connected extensions, wherein the inward extensions are adapted to guide medical instruments within the flexure.
